(19) United States
(12) Patent Application Publication
(10) Pub. No.: US 2025/0235252 A1
(43) Pub. Date: Jul. 24, 2025

(54) ACTIVELY STEERABLE GUIDEWIRE WITH TOME

(71) Applicants: Boston Scientific Medical Device Limited, Galway (IE); Boston Scientific Scimed, Inc., Maple Grove, MN (US)

(72) Inventors: Deepak Kumar Sharma, Muzaffarnagar (IN); John Thomas Favreau, Charlton, MA (US)

(73) Assignees: Boston Scientific Medical Device Limited, Galway (IE); Boston Scientific Scimed, Inc., Maple Grove, MN (US)

(21) Appl. No.: 19/029,350

(22) Filed: Jan. 17, 2025

Related U.S. Application Data
(60) Provisional application No. 63/622,901, filed on Jan. 19, 2024.

(51) Int. Cl. A61B 18/00; A61B 1/00

(52) U.S. Cl. CPC A61B 18/00; A61B 1/00066; A61B 1/00119; A61B 2018/00077; A61B 2018/00083; A61B 2018/00095; A61B 2018/00101; A61B 2018/00184; A61B 2018/00601; A61B 2018/00982

(57) ABSTRACT

Devices, systems, and methods for a combination guidewire and tome shaft comprising three wires. The upper wire is manipulated to actively steer the shaft, while the lower wire creates a bowed region for cutting tissue. Insulating sheaths may cover the end of the shaft other than the bowed region of the lower wire. A voltage source allows electrification of the lower wire during cutting.

### CROSS-REFERENCE TO RELATED APPLICATIONS

The application claims the benefit of U.S. Provisional Patent Application Ser. No. 63/622,901 filed on Jan. 19, 2024, the disclosure of which is incorporated herein by reference.

### FIELD

The disclosure relates generally to devices, systems, and methods for steerable medical devices. More specifically, aspects of the disclosure pertain to devices, systems, and/or methods for surgical tomes and steerable guidewires.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages.

An operator may insert tools through a working channel of a medical device during procedures such as endoscopy, colonoscopy, and/or Endoscopic Retrograde Cholangiopancreatography (“ERCP”). In such procedures, an operator may desire to position an instrument or device (such as a tool) at a particular position, at or distal to a distal tip of the medical device. In order to position an instrument or device in a desired position, an operator may utilize a guidewire. An operator may pass a guidewire through a working channel of an endoscope (or through a lumen of another device, such as a guide catheter or tome), and then use the guidewire in order to position the instrument or device. For example, an operator may slide the instrument, such as a snare, or device, such as a stent, over the guidewire. Guidewires may also facilitate exchange of instruments and devices in an efficient fashion. An operator may remove and replace an instrument or device, guiding the new instrument or device to the same location by using the guidewire.

Guidewires may also be utilized in vascular applications. For example, an operator may utilize a guidewire in order to position a stent, catheter, or other structure. Guidewires may similarly be used in urological procedures (e.g., to place a ureteral stent or a catheter). Positioning a guidewire in a desired position is important for each of these procedures, as well as others involving guidewires. Therefore, a need exists for devices, systems, and methods for steerable medical devices, including steerable guidewires.

### SUMMARY

This disclosure provides design, material, manufacturing method, and use alternatives for medical devices and medical systems. In a first example, a combination guidewire and tome shaft for an endoscopic medical device includes a middle wire, an upper wire, and a lower wire. The middle wire has an upper surface, a lower surface, and a distal region defined by an eccentric cut in the upper surface and ending at a distal tip. The upper wire is attached along the upper surface of the middle wire and has a distal region that ends in a distal tip that is attached to the distal tip of the middle wire. The lower wire is attached along the lower surface of the middle wire, has a distal region defined by an eccentric cut in the lower wire, and ends in a distal tip that is attached to the middle wire at a point proximal of the distal region of the middle wire. The upper wire is movable relative to the middle wire to actively steer the shaft. The lower wire is movable relative to the middle wire to create a bowed region suitable for cutting tissue during an endoscopic procedure.

Alternatively or additionally to any of the examples above, the distal region of the upper wire can be defined by an eccentric cut in the upper wire.

Alternatively or additionally to any of the examples above, the upper wire can include a lower surface, the middle wire can be attached to the upper wire along the lower surface, and the eccentric cut in the upper wire can be in the lower surface of the upper wire.

Alternatively or additionally to any of the examples above, the lower wire can include an upper surface, the middle wire can be attached to the lower wire along the upper surface, and the eccentric cut in the lower wire can be in the upper surface of the lower wire.

Alternatively or additionally to any of the examples above, the combination guidewire and tome shaft can further include a proximal sheath surrounding the upper, middle and lower wires proximal of the distal region of the lower wire.

Alternatively or additionally to any of the examples above, the combination guidewire and tome shaft can further include a distal sheath surrounding the upper and middle wires, the distal sheath running alongside but not covering the distal region of the lower wire.

Alternatively or additionally to any of the examples above, the distal sheath can cover the upper and middle wires to the distal tips of the upper and middle wires.

Alternatively or additionally to any of the examples above, the sheath and/or sheaths can comprise an insulating material.

Alternatively or additionally to any of the examples above, the insulating material can be an elastomer.

Alternatively or additionally to any of the examples above, the insulating material can be polyether block amide.

Alternatively or additionally to any of the examples above, the wires can be made of stainless steel.

Alternatively or additionally to any of the examples above, the wires can be made of nitinol.

In another example, an endoscopic medical device, can include a combination guidewire and tome shaft of any of the examples above, and a handle including a handle body defining a channel, the channel receiving the combination guidewire and tome shaft.

Alternatively or additionally to any of the examples above, the handle can open to allow insertion of an endoscopic tool guided by the shaft.

Alternatively or additionally to any of the examples above, the medical device can further include a voltage source in electrical communication with the lower wire of the shaft to electrify the bowed region of the shaft when cutting tissue.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts an exemplary steerable medical device;

FIG. 2 depicts the distal end of wires comprising an exemplary shaft of a medical device;

FIG. 2A depicts the distal end of the upper wire of the exemplary shaft of FIG. 2;

FIG. 2B depicts the distal end of the middle wire of the exemplary shaft of FIG. 2;

FIG. 2C depicts the distal end of the lower wire of the exemplary shaft of FIG. 2;

FIG. 3A depicts the distal end of an exemplary shaft including sheaths;

FIG. 3B depicts the shaft of FIG. 3A with a deployed bowed region to act as a tome;

FIG. 3C depicts the shaft of FIG. 3A actively steered;

FIG. 4A depicts the distal end of an exemplary shaft including fluorescence; and

FIG. 4B depicts the distal end of an exemplary shaft including notches.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

As used in this disclosure, the terms “upper” and “lower” are relative terms used to differentiate between opposite directions, not the overall orientation of the device. One of ordinary skill will recognize that a device could operate, for example, with the “uppermost” component at the bottom and the “lowermost” component at the top (i.e., “upside-down” when comparing the device operation to the terms), or in any other absolute orientation relative to the user and/or gravity.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

A medical device may include a handle and a steerable shaft. The shaft may include, for example, a steerable guidewire. Although the disclosure may reference guidewires used in conjunction with endoscopic, colonoscopic, and/or ERCP procedures, it will be appreciated that the guidewires may also be utilized for other types of procedures, including vascular or urologic procedures. The medical devices disclosed herein may have three degrees of freedom for movement. The shaft may be articulated (i.e. bent or deflected) in one or more directions. Furthermore, the shaft may be rotatable and movable proximally and distally. The handle may be removable from the shaft so as to facilitate use of the guidewire to guide tools or medical devices after the guidewire is positioned. The shaft may include two lumens with one or two articulation wires, either or both of which may be utilized to achieve a desired steering/articulation of the shaft. The medical devices disclosed herein may overcome limitations to accessing complex anatomies and resolve or assist with navigation challenges. The disclosed medical devices may eliminate the high learning curve associated with other devices. The disclosed medical devices may supplant the multiple guidewires that are currently used, depending on anatomy and the type of medical procedure, thereby streamlining the types of guidewires that a medical provider must stock and limiting potential for confusion surrounding different types of guidewires. The disclosed devices may be compatible with robotics platforms. Furthermore, procedures may require accessing a first location in an anatomy, followed by one or more further locations, location within the same area of the anatomy. The disclosed, steerable devices may allow access to multiple locations within the area of the anatomy, without inserting and removing a first guidewire and re-navigating to additional locations. Thus, the disclosed devices may avoid needing multiple guidewires to be placed in multiple locations in the anatomy. Any of the handles disclosed herein may be removed and/or replaced with the same handle or with an alternate handle. Similarly, the shafts disclosed herein may be utilized with any of the disclosed handles. Thus, during a procedure, an operator may use different handles at different portions of the procedure or may use a shaft without a handle.

Although the disclosure may refer at different points to one of a duodenoscope or an endoscope, it will be appreciated that, unless otherwise specified, duoendoscopes, endoscopes, colonoscopes, ureteroscopes, bronchoscopes, laparoscopes, sheaths, catheters, or any other suitable delivery device or medical device may be used in connection with the guidewires and methods of the disclosure.

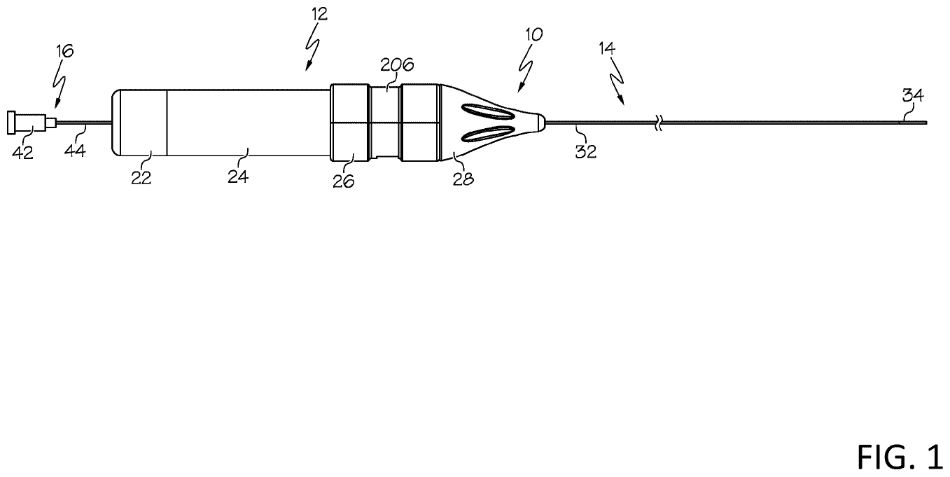

As shown in FIG. 1, medical device 10 may include a handle 12 and a shaft 14. Shaft 14 may have any suitable outer width (e.g., diameter). For example, an outer width (e.g., diameter) of shaft 14 may be similar to or the same as a typical guidewire. Exemplary outer diameters may range from approximately .010 inches to approximately . 04 inches or more particularly from approximately .014 inches to approximately .032 inches. An outer diameter may be constant along a length of shaft 14 or may vary.

Handle 12 may include various bodies, including a cap 22, a plunger 24, a slider 26, and a collet 28. Shaft 14 may include a proximal section 32 and a distal section 34 (distal section 34 being distal to proximal section 32).

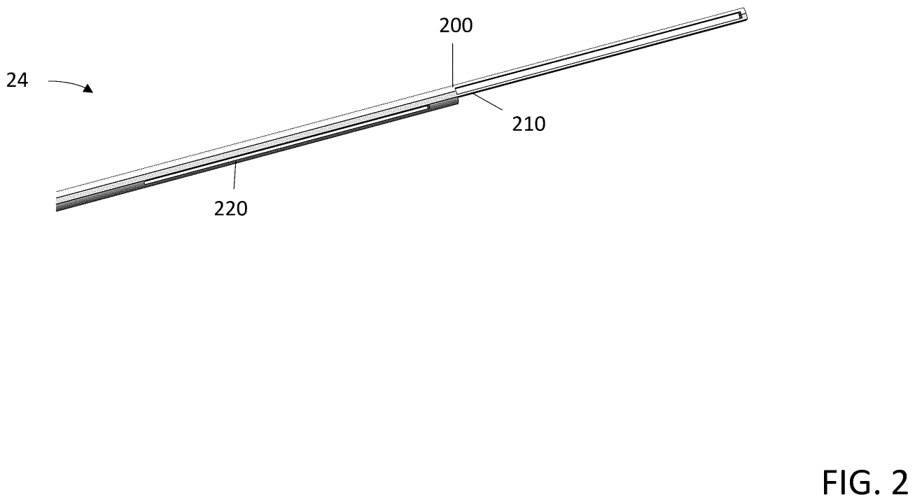

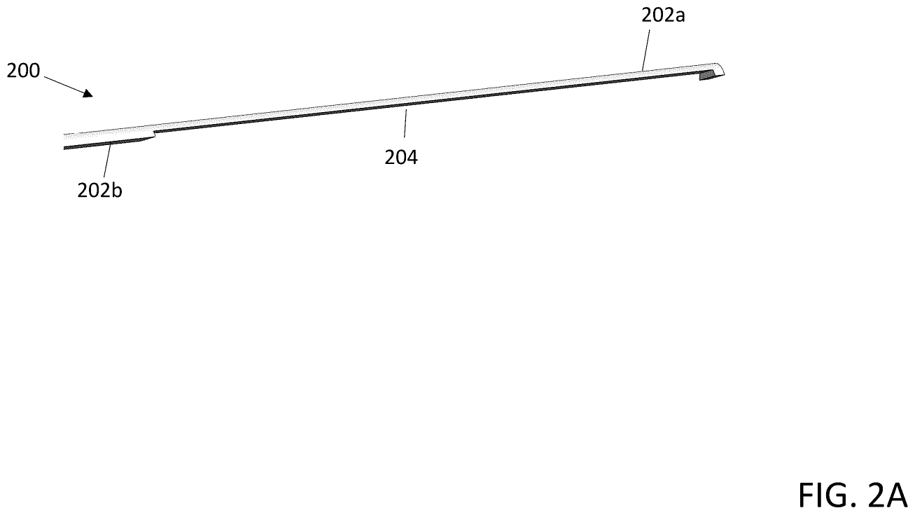

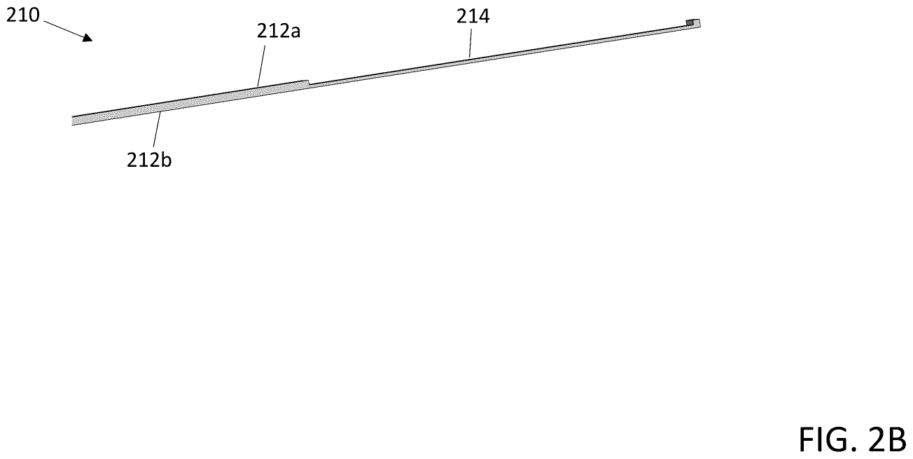

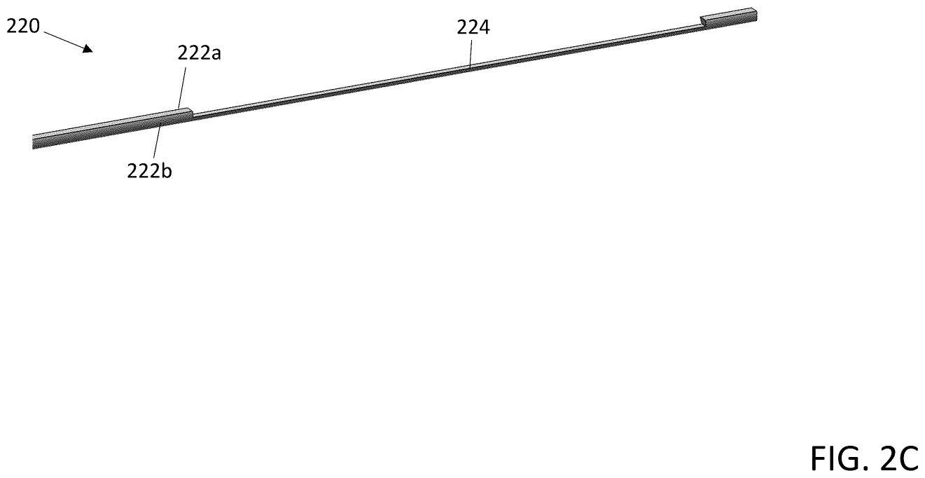

The shaft 14 includes multiple strands representing both a guidewire and a tome, as shown in more detail in FIGS. 2-2C. FIG. 2 shows distal section 34 of the shaft 14, comprising the upper wire 200, the middle wire 210, and the lower wire 220. FIG. 2A illustrates the upper wire 200 is D-shaped, having an upper curved surface 202a and a lower flat surface 202b. An eccentric cut 204 is made in the flat surface 202b near the distal end, such that the cut-out region of the upper wire 200 faces the middle wire 210.

The middle wire 210 (shown in FIG. 2B) includes an upper flat surface 212a facing the lower flat surface 202b of the upper wire 200. The upper and middle wires 200, 210 are joined at their respective flat surfaces 202b, 212a. An eccentric cut 214 is made in the upper flat surface 212a near the distal end, such that the cut-out region of the middle wire 210 faces the upper wire 200. As shown in FIG. 2, the length of the cuts 204 and 214 may be the same length and positioned so as to form a rectangular cut-out region bordered by both wires 200 and 210. One of ordinary skill will recognize that, in some embodiments, the eccentric cuts in the two wires may not precisely correspond; their length or positions may vary one from the other as required to carry out the guidewire and tome functions of the device as herein described.

The lower wire 220 (FIG. 2C) is D-shaped, having a flat upper surface 222a joined to the flat lower surface 212b of the middle wire 210 (FIG. 2). As shown, the lower wire 220 may be shorter than the upper and middle wires 200,210 such that the distal tip of the lower wire 220 is attached along the length of the middle wire 210. An eccentric cut 224 is made in the upper flat surface 222a, so that the cut-out region of the lower wire 220 faces the middle wire 210.

Each of the wires 200, 210, and 220 may be made of the same material or different materials. The wires may be made of any material with sufficient ductility, electroconductivity, flexibility and resilience to allow for the guidewire and tome functions described herein. For example, the wires may be made of nitinol, stainless steel, or another metal. As only the lower wire 220 is energized for cutting purposes as described above, the material of the lower wire 220 may have a higher electroconductivity than the material of the upper and/or middle wires 200 and 210.

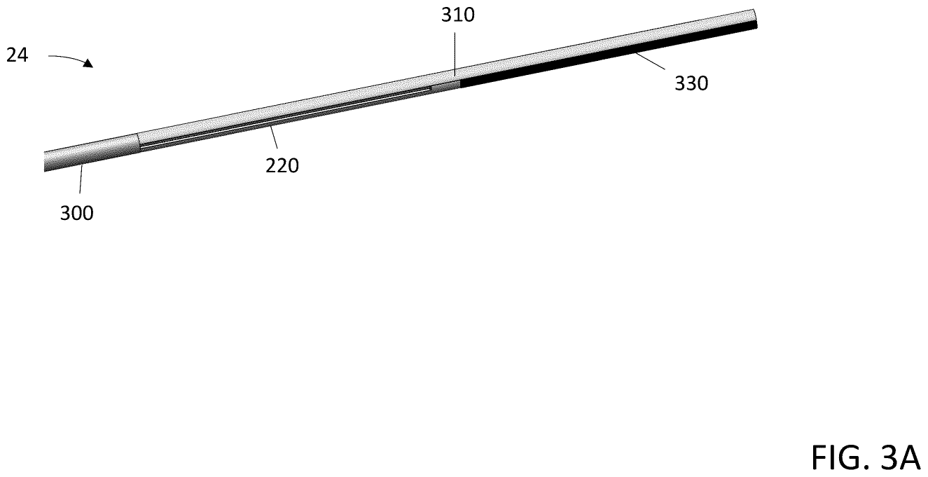

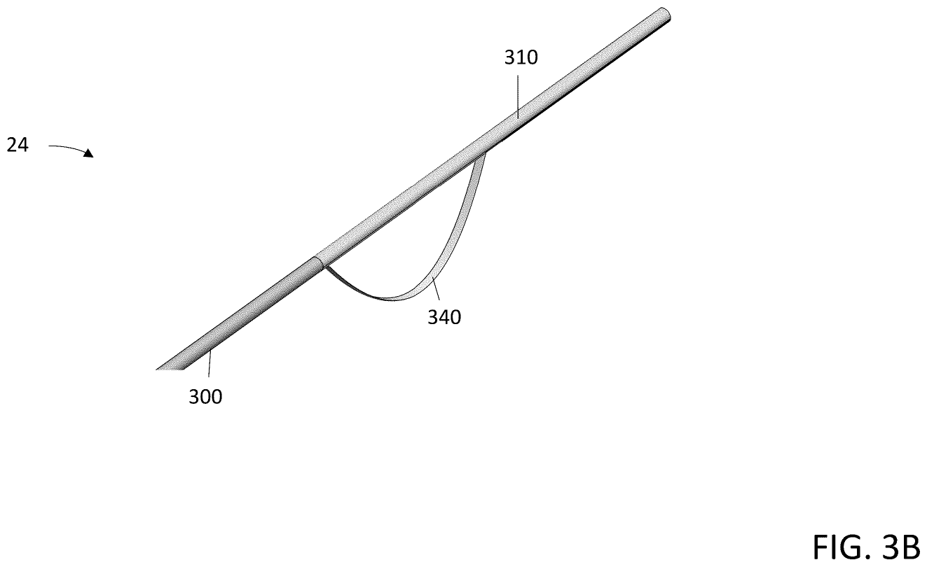

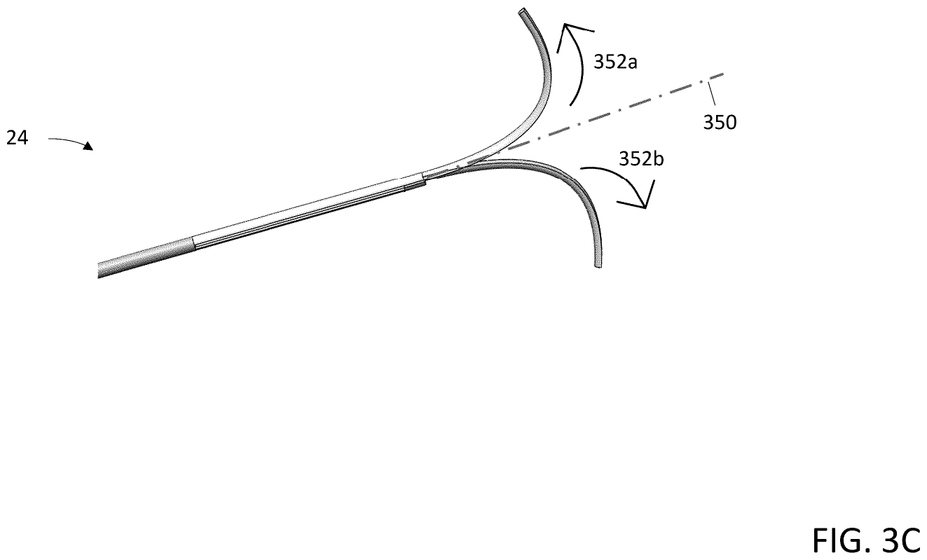

As shown in FIG. 3A, the wires 200, 210, and 220 of the shaft are surrounded by sheath portions to provide insulation during endoscopic procedures. A full sheath 300 encloses the main body of the shaft 24 proximal of the eccentric cut 224. A partial sheath 310 distal of the full sheath 300 covers only the upper and middle wires 200, 210 while leaving the lower wire 220 exposed to act as a cut wire for cannulation as further described below. Past the distal tip of the lower wire 220, a supplemental end wire 330 attaches to the middle wire 210 opposite the upper wire 200 to round out the shape of the shaft 24.

In some implementations, the sheaths 300 and 310 may be an insulative elastomer, such as a polyether block amide. Other insulative materials, such as insulative elastomers or flexible composites, will be recognized as appropriate alternatives based on their properties. The supplemental end wire 330 may any appropriate material, for example, a tungsten alloy.

When the user pushes a proximal portion of the lower wire 220 towards the distal end, it bows as shown in FIG. 3B. The bowed region 340 can then be used as a sphincterotome or other cutting tool needed during the endoscopic procedure. While the rest of the shaft 24 is insulated, the bowed region 340 is not. Current run through the wires can be used to provide cauterizing energy for cannulation using the bowed region 340. When the cutting is complete, the lower wire 220 can be pulled towards the proximal end to retract the region 340 along shaft.

The upper wire 200 can be pushed or pulled in order to curve the shaft upwards or downwards, as shown in FIG. 3C. Here, relative to longitudinal axis 350, the shaft 24 can be curved upwards according to the arrow 352a by pulling the upper wire 200 towards the proximal end. The shaft 24 can instead be curved downwards, according to the arrow 352b, by pushing the upper wire 200 towards the distal end. Pushing or pulling in the opposite direction can then be used to return the shaft 24 to a straight orientation according to the axis 350.

The shaft 24 is an actively steerable guidewire by means of the upper wire 200 as illustrated. Other tools may be introduced and removed using the shaft 24 as a guidewire. Because the region 340 is an integral part of the shaft 24, the shaft acts both as a guidewire and a cutter; a separate tome device is not needed.

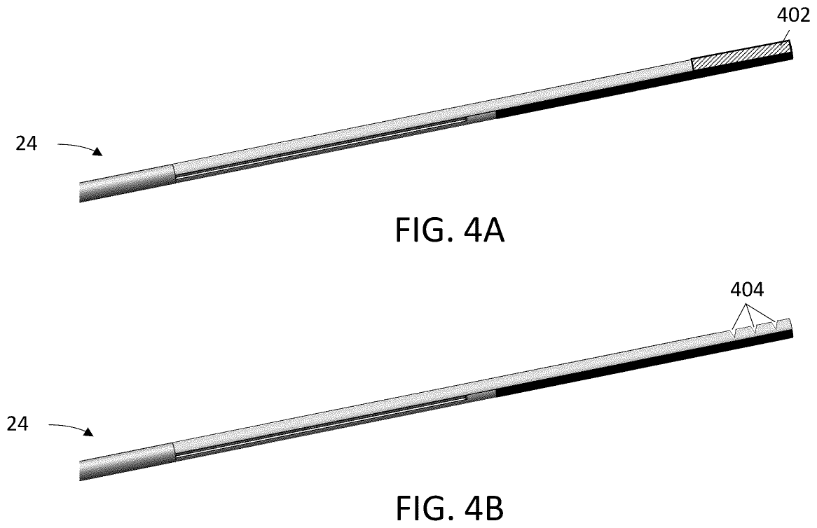

One of ordinary skill will recognize other variations of the shaft 24 that are appropriate due to its role as a guidewire and as a tome during endoscopic procedures. As shown in FIG. 4A, the shaft may include a fluorescent region 402 for better visibility when actively steering the device. Features in the shape of the shaft, such as notches 404, may be added to assist in identification of the shaft's orientation when monitored by ultrasound sensors. Other known features for guidewires used in endoscopy may be similarly added to this device.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A combination guidewire and tome shaft for an endoscopic medical device, comprising:
a middle wire having an upper surface, a lower surface, and a distal region defined by an eccentric cut in the upper surface and ending at a distal tip;
an upper wire attached along the upper surface of the middle wire, the upper wire having a distal region defined by an eccentric cut in the upper wire and ending in a distal tip that is attached to the distal tip of the middle wire; and
a lower wire attached along the lower surface of the middle wire, the lower wire having a distal region defined by an eccentric cut in the lower wire and ending in a distal tip that is attached to the middle wire at a point proximal of the distal region of the middle wire;
wherein the upper wire is movable relative to the middle wire to actively steer the shaft; and
wherein the lower wire is movable relative to the middle wire to create a bowed region suitable for cutting tissue during an endoscopic procedure.

2. The shaft of claim 1, wherein the distal region of the upper wire is defined by an eccentric cut in the upper wire.

3. The shaft of claim 2, wherein the upper wire comprises a lower surface, the middle wire is attached to the upper wire along the lower surface, and the eccentric cut in the upper wire is in the lower surface of the upper wire.

4. The shaft of claim 1, wherein the lower wire comprises an upper surface, the middle wire is attached to the lower wire along the upper surface, and the eccentric cut in the lower wire is in the upper surface of the lower wire.

5. The shaft of claim 1, further comprising a proximal sheath surrounding the upper, middle and lower wires proximal of the distal region of the lower wire.

6. The shaft of claim 5, the proximal sheath comprising an insulating material.

7. The shaft of claim 6, wherein the insulating material is an elastomer.

8. The shaft of claim 7, wherein the insulating material is polyether block amide.

9. The shaft of claim 1, further comprising a distal sheath surrounding the upper and middle wires, the distal sheath running alongside but not covering the distal region of the lower wire.

10. The shaft of claim 9, wherein the distal sheath covers the upper and middle wires to the distal tips of the upper and middle wires.

11. The shaft of claim 9, the distal sheath comprising an insulating material.

12. The shaft of claim 11, wherein the insulating material is an elastomer.

13. The shaft of claim 12, wherein the insulating material is polyether block amide.

14. The shaft of claim 1, wherein the wires are made of stainless steel.

15. The shaft of claim 1, wherein the wires are made of nitinol.

16. An endoscopic medical device, comprising:
a combination guidewire and tome shaft comprising:
a middle wire having an upper surface, a lower surface, and a distal region defined by an eccentric cut in the upper surface and ending at a distal tip,
an upper wire attached along the upper surface of the middle wire, the upper wire having a distal region defined by an eccentric cut in the upper wire and ending in a distal tip that is attached to the distal tip of the middle wire, and
a lower wire attached along the lower surface of the middle wire, the lower wire having a distal region defined by an eccentric cut in the lower wire and ending in a distal tip that is attached to the middle wire at a point proximal of the distal region of the middle wire,
wherein the upper wire is movable relative to the middle wire to actively steer the shaft, and
wherein the lower wire is movable relative to the middle wire to create a bowed region suitable for cutting tissue during an endoscopic procedure; and
a handle comprising a handle body defining a channel, the channel receiving the combination guidewire and tome shaft.

17. The endoscopic medical device of claim 16, wherein the handle opens to allow insertion of an endoscopic tool guided by the shaft.

18. The endoscopic medical device of claim 16, further comprising a voltage source in electrical communication with the lower wire of the shaft to electrify the bowed region of the shaft when cutting tissue.

19. The endoscopic medical device of claim 16, wherein the wires of the combination guidewire and tome shaft are made of stainless steel.

20. The endoscopic medical device of claim 16, wherein the wires of the combination guidewire and tome shaft are made of nitinol.
